(19) United States
(12) Patent Application Publication
(10) Pub. No.: US 2010/0215747 A1
(43) Pub. Date: Aug. 26, 2010

(54) NANOPARTICLES COMPRISING IONIZABLE, POORLY WATER SOLUBLE CELLULOSIC POLYMERS

(76) Inventors: Corey Jay Bloom, Bend, OR (US); Marshall David Crew, Bend, OR (US); Dwayne T. Friesen, Bend, OR (US); Warren Kenyon Miller, Bend, OR (US); Michael Mark Morgen, Bend, OR (US); Daniel Tod Smithey, Bend, OR (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/451,813

(22) PCT Filed: Jun. 30, 2008
(86) PCT No.: PCT/IB2008/001801

§ 371 Date: Apr. 21, 2010

Related U.S. Application Data
(60) Provisional application No. 60/949,525, filed on Jul. 13, 2007.

(51) Int. Cl. A61K 9/14; A61K 47/34; A61K 47/38; A61P 43/00

(52) U.S. Cl. 424/487; 424/488; 424/499; 514/772.4; 514/781

(57) ABSTRACT

A pharmaceutical composition comprises nanoparticles comprising ionizable, poorly water soluble cellulosic polymers.

### BACKGROUND OF THE INVENTION

The present invention relates to nanoparticles comprising a poorly water soluble drug and ionizable, poorly water soluble cellulosic polymers.

A variety of approaches have been taken to formulate drugs as nanoparticles. One approach is to decrease the size of crystalline drug by grinding or milling the drug in the presence of a surface modifier. See, e.g., U.S. Pat. No. 5,145,684. Another approach to forming nanoparticles is to precipitate the drug in the presence of a film forming material such as a polymer. See, e.g., U.S. Pat. No. 5,118,528.

Nanoparticles have been formed from a variety of polymers. Bodmeier and Chen (J. Controlled Release, 12, (1990) 223-233) disclose forming nanoparticles with a non-ionizable polymer such as ethylcellulose or with a cationic polymer such as Eudragit RL or RS. Gurny, et al., US Patent Application 2004/0018236 A1 disclose nanoparticles comprising drugs having low water solubility and anionic polymers. The polymer is enteric, i.e., resistant to gastric juices and soluble in intestinal juices. Exemplary enteric polymers include Eudragit L and Eudragit S, polyvinyl acetate phthalate, hydroxypropylmethyl cellulose acetate succinate, hydroxypropylmethyl cellulose phthalate, cellulose acetate phthalate, and cellulose acetate trimellitate. Nanoparticles made with Eudragit L and Eudragit S are exemplified.

### BRIEF SUMMARY OF THE INVENTION

A pharmaceutical composition comprising nanoparticles comprises a drug and an ionizable, cellulosic polymer. The drug is a poorly water soluble drug having a solubility in water of less than 5 mg/mL over the pH range of 6.5 to 7.5 at 25° C. At least 90 wt % of the drug in the nanoparticles is non-crystalline. The ionizable cellulosic polymer is poorly water soluble, so that when the polymer is administered alone at a solids concentration of 0.2 mg/mL to a phosphate buffered saline (PBS) solution consisting of an aqueous solution of 20 mM sodium phosphate (Na₂HPO₄), 47 mM potassium phosphate (KH₂PO₄), 87 mM NaCl, and 0.2 mM KCl, adjusted to pH 6.5 with NaOH, the polymer has a solubility 0.1 mg/mL or less. The nanoparticles have an average size of less than 500 nm. The drug and the polymer constitute at least 60 wt % of the nanoparticles.

In one embodiment, the polymer, when equilibrated at 85% relative humidity at 25° C., absorbs less than 7.5 wt % water.

In another embodiment, the drug and the polymer are molecularly interdispersed within one another.

In another embodiment, the nanoparticles comprise a solid solution of the drug and the polymer.

In another embodiment, the nanoparticles have an average size of less than 300 nm.

In another embodiment, the nanoparticles have an average size of less than 150 nm.

In another embodiment, the nanoparticles have a zeta potential with an absolute value of greater than 10 mV.

In another embodiment, the drug and the polymer constitute at least 80 wt % of the nanoparticles.

In another embodiment, the nanoparticles consist essentially of the drug and the polymer.

In another embodiment, the nanoparticles are substantially free from a surfactant.

In another embodiment, the polymer comprises an ether-linked ethyl substituent and an ether- or ester-linked ionizable substituent.

In another embodiment, the polymer is selected from the group consisting of ethylcellulose succinate, ethylcellulose phthalate, and ethylcellulose trimellitate.

In another embodiment, the polymer is selected from the group consisting of hydroxypropyl methylcellulose acetate succinate, hydroxypropyl methylcellulose propionate succinate, hydroxypropyl methylcellulose butyrate succinate, hydroxypropyl methylcellulose acetate phthalate, hydroxypropyl methylcellulose propionate phthalate, hydroxypropyl methylcellulose butyrate phthalate, hydroxypropyl methylcellulose acetate trimellitate, hydroxypropyl methylcellulose propionate trimellitate, hydroxypropyl methylcellulose butyrate trimellitate, carboxymethyl ethylcellulose, cellulose acetate propionate succinate, cellulose acetate succinate, cellulose propionate succinate, cellulose butyrate succinate, cellulose acetate phthalate, cellulose propionate phthalate, cellulose butyrate phthalate, cellulose acetate trimellitate, cellulose propionate trimellitate, cellulose butyrate trimellitate, carboxymethylcellulose acetate butyrate, carboxymethylcellulose acetate, carboxymethylcellulose propionate, and carboxymethylcellulose butyrate.

In another embodiment, the polymer is selected from the group consisting of hydroxypropyl methyl cellulose acetate succinate (HPMCAS), hydroxypropyl methyl cellulose acetate phthalate (HPMCAP), hydroxypropyl methyl cellulose acetate trimellitate (HPMCAT), ethylcellulose succinate (ECS), ethylcellulose phthalate (ECP), ethylcellulose trimellitate (ECT), carboxymethyl ethylcellulose (CMEC), cellulose acetate propionate succinate (CAPrS), cellulose acetate succinate (CAS), cellulose propionate succinate (CPrS), cellulose acetate phthalate (CAP), and carboxymethylcellulose acetate butyrate (CMCAB).

In another embodiment, the solubility of the polymer is less than 0.07 mg/mL.

In another embodiment, the solubility of the polymer is less than 0.05 mg/mL.

In another embodiment, the polymer has a degree of substitution of ionizable substituents of at least 0.03.

In another embodiment, the water solubility of the drug is less than 1 mg/mL.

In another embodiment, the composition further comprises a matrix material, wherein the nanoparticles are entrapped in the matrix material.

In another embodiment, the matrix material is selected from the group consisting of polyvinyl pyrrolidone (PVP), trehalose, hydroxypropyl methyl cellulose (HPMC), hydroxypropyl cellulose (HPC), casein, caseinate, albumin, gelatin, acacia, lactose, mannitol, pharmaceutically acceptable forms thereof, and mixtures thereof.

Because the nanoparticles are formed from a poorly aqueous soluble anionic polymer, the stability of the non-crystalline drug and the suspension stability of the nanoparticle are both addressed, resulting in nanoparticles with improved performance and stability.

First, the poorly aqueous soluble polymer used in the nanoparticles stabilizes the poorly water soluble drug in the sense of reducing the rate of crystallization of the drug in the solid state and while in suspension. In addition, because the non-ionizable polymer is poorly aqueous soluble at physiological pH, the nanoparticles maintain the drug within a solid (or at least undissolved) polymer matrix when the nanoparticles are suspended in an aqueous solution, further preventing or reducing crystallization of the drug. It is well known that the non-crystalline form of a low-solubility drug provides a greater aqueous concentration of drug relative to the crystalline form of the drug when administered to an aqueous use environment. However, it is also well known that when the drug is not stabilized in the non-crystalline form, the drug rapidly converts to the crystalline form in the use environment. See, for example, Hancock and Parks (Pharmaceutical Research, Vol. 17, No. 4, 2000). Thus, the poorly aqueous soluble non-ionizable polymer is selected to maintain the stability of the non-crystalline drug in the nanoparticle and while suspended in an aqueous solution, resulting in an enhanced concentration of free drug when the nanoparticle is administered to an aqueous use environment.

Second, the anionic cellulosic polymer provides a charge when the nanoparticles are suspended in an aqueous use environment, thus reducing or eliminating agglomeration of the nanoparticles. The anionic cellulosic polymer also results in improved re-suspendability of solid compositions containing nanoparticles relative to surfactant-based and neutral polymer-based stabilizers: solid compositions of the invention resuspend nanoparticles when administered to an aqueous solution.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1. Cryo-TEM photomicrograph of the nanoparticles of Example 16.

FIG. 2. Powder X-Ray Diffraction (PXRD) Diffractogram of Example 16.

### DETAILED DESCRIPTION OF PREFERRED EMBODIMENTS

The nanoparticles of the present invention comprise a poorly water soluble drug and an ionizable, poorly water soluble cellulosic polymer. At least 90 wt % of the drug in the nanoparticle is non-crystalline. The nature of the nanoparticles, suitable polymers and drugs, and methods for making nanoparticles are described in detail below.

### Nanoparticles

The nanoparticles are small particles of drug and the polymer, with each nanoparticle containing the drug and the ionizable polymer. While the bulk drug prior to formation of the nanoparticles may be either crystalline or non-crystalline, at least 90 wt % of the drug in the nanoparticles is non-crystalline. The term “crystalline,” as used herein, means a particular solid form of a compound that exhibits long-range order in three dimensions. “Non-crystalline” refers to material that does not have long-range three-dimensional order, and is intended to include not only material which has essentially no order, but also material which may have some small degree of order, but the order is in less than three dimensions and/or is only over short distances (e.g., only over a few molecules). Another term for a non-crystalline form of a material is the “amorphous” form of the material. As previously discussed, the non-crystalline form of a low-solubility drug is preferred as it provides a greater aqueous concentration of drug relative to the crystalline form of the drug in an aqueous use environment. Preferably at least about 95 wt % of the drug in the nanoparticle is non-crystalline; in other words, the amount of drug in crystalline form does not exceed about 5 wt %. Non-crystalline drug may be characterized by the absence of a melt temperature, or the absence of diffraction peaks when analyzed by Powder X-Ray Diffraction (PXRD). Amounts of crystalline drug may be measured by PXRD, by Differential Scanning Calorimetry (DSC), by solid state nuclear magnetic resonance (NMR), or by any other known quantitative measurement.

The non-crystalline drug in the nanoparticle can exist as a pure phase, as a solid solution of drug homogeneously distributed throughout the polymer, or any combination of these states or those states that lie between them. In one embodiment, the drug and polymer are interdispersed within one another at the molecular level. Preferably, at least a portion of the drug and the polymer is present in the nanoparticle in the form of a solid solution. The solid solution may be thermodynamically stable, in which the drug is present at less than the solubility limit of the drug in the polymer, or may be a supersaturated solid solution in which the drug exceeds its solubility limit in the polymer. Preferably essentially all of the drug and the polymer are present as a solid solution.

The nanoparticles can exist in a number of different configurations. In one embodiment, the nanoparticles comprise a core, the core comprising the non-crystalline drug and the polymer. As used herein, the term “core” refers to the interior portion of the nanoparticle. The nanoparticles also have a “surface portion,” meaning the outside or exterior portion of the nanoparticle. Thus, the nanoparticles consist of a core (i.e., the interior portion) and a surface portion. In some embodiments, described herein below, materials may be adsorbed to the surface portion of the nanoparticle. Materials adsorbed to the surface portion of the nanoparticle are considered part of the nanoparticle, but are distinguishable from the core of the nanoparticle. Methods to distinguish materials present in the core versus materials adsorbed to the surface portion of the nanoparticle include (1) thermal methods, such as differential scanning calorimetry (DSC); (2) spectroscopic methods, such as X-ray photoelectron spectroscopy (XPS), transmission electron microscopy (TEM) with energy dispersive X-ray (EDX) analysis, Fourier transform infra red (FTIR) analysis, and Raman spectroscopy; (3) chromatographic techniques, such as high performance liquid chromatography (HPLC), and gel-permeation chromatography (GPC); and (4) other techniques known in the art.

In one embodiment, the non-crystalline drug and the polymer constitute at least 60 wt % of the core, more preferably at least 80 wt % of the core. In another embodiment, the core consists essentially of the non-crystalline drug and the polymer.

The non-crystalline drug present in the core can exist in non-crystalline pure drug domains, as a thermodynamically stable solid solution of non-crystalline drug homogeneously distributed throughout the polymer, as a supersaturated solid solution of non-crystalline drug homogeneously distributed throughout the polymer, or any combination of these states or those states that lie between them. When the glass-transition temperature (T_{g}) of the non-crystalline drug is different from the T_{g} of the pure polymer by at least about 20° C., the core may exhibit a T_{g} that is between the T_{g} of pure non-crystalline drug or pure polymer. Preferably, less than 20 wt % of the drug is present in non-crystalline drug domains, with the remaining drug homogeneously distributed throughout the polymer.

The drug and polymer are collectively present in the nanoparticle in an amount ranging from 60 wt % to 100 wt %. Preferably, the drug and polymer constitute at least 70 wt %, more preferably at least 80 wt %, and even more preferably at least 90 wt % of the nanoparticle. In one embodiment, the nanoparticles consist essentially of the drug and polymer. By “consist essentially of” is meant that the nanoparticle contains less than 1 wt % of any other excipients and that any such excipients have no affect on the performance or properties of the nanoparticle.

The amount of drug in the nanoparticle may range from 0.1 wt % to 99 wt %. The physical stability of the non-crystalline drug (meaning its tendency to crystallize) within the nanoparticle tends to improve as the amount of drug in the nanoparticle decreases. Accordingly, it is preferred that the amount of drug in the nanoparticle is less than about 70 wt %, and more preferably less than about 60 wt %.

The weight ratio of drug to polymer may vary from 100:1 (100 parts drug to 1 part polymer) to 0.01:1 (1 part drug to 100 parts polymer). The weight ratio of drug to polymer may range from 0.05:1 to 3:1, from 0.1:1 to 2:1, or from 0.2:1 to 1.5:1. In one embodiment, the nanoparticles have the following amounts of drug and polymer: 5 to 60 wt % drug and 95 to 40 wt % polymer, more preferably 10 to 50 wt % drug and 90 to 50 wt % polymer.

The nanoparticle is preferably substantially free from surfactants. By a “surfactant” is meant a surface active material having a hydrophobic portion and a hydrophilic portion, and which is soluble in the use environment. By substantially “free from” is meant that the amount of surfactant present in the composition is less than 0.1 wt %. Preferably, the amount of the surfactant present in the nanoparticles is less than the detection limit. As discussed above, surfactants can be poorly tolerated in vivo, and thus it is preferred to avoid their use in the instant nanoparticles.

The nanoparticles are ionized when present in an aqueous use environment. It is believed that stability of the nanoparticles, in the sense of not aggregating or flocculating, is related, in part, to the amount of electric charge on the nanoparticle. The charge may be either positive or negative. An indirect measure of charge is zeta potential. The nanoparticles preferably have a zeta potential of less than −10 mV or greater than +10 mV (that is, the absolute value of the zeta potential is greater than 10 mV). Preferably, to reduce aggregation, the absolute value of the zeta potential is at least 20 mV, more preferably at least 30 mV, and even more preferably at least 40 mV. Zeta potential is typically calculated from the electrophoretic mobility measured by light scattering, R. J. Hunter, Zeta Potential in Colloid Science. Principles and Applications, Academic Press, 1981. Zeta potential may be measured using any number of commercially-available instruments, such as Brookhaven Instruments Corp. ZetaPals zeta potential analyzer.

The average size of the nanoparticles in suspension is less than 500 nm. In suspension, by “average size” is meant the effective cumulant diameter as measured by dynamic light scattering, using for example, Brookhaven Instruments' 90Plus particle sizing instrument. By “size” is meant the diameter for spherical particles, or the maximum diameter for non-spherical particles. Preferably, the average size of the nanoparticles is less than 400 nm, more preferably less 300 nm, and most preferably less than 200 nm.

The width of the particle size distribution in suspension is given by the “polydispersity” of the particles, which is defined as the relative variance in the correlation decay rate distribution, as is known by one skilled in the art. See B. J. Fisken, “Revisiting the method of cumulants for the analysis of dynamic light-scattering data,” Applied Optics, 40(24), 4087-4091 (2001) for a discussion of cumulant diameter and polydispersity. Preferably, the polydispersity of the nanoparticles is less than 0.5. More preferably, the polydispersity of the nanoparticles is less than about 0.3. In one embodiment, the average size of the nanoparticles is less than 500 nm with a polydispersity of 0.5 or less. In another embodiment, the average size of the nanoparticles is less than 300 nm with a polydispersity of 0.5 or less.

### Ionizable Cellulosic Polymers

The term “polymer” is used conventionally, meaning a compound that is made of monomers connected together to form a larger molecule. The polymer should be inert, in the sense that it does not chemically react with the drug in an adverse manner. The molecular weight may vary, and in general is less than 200,000 daltons.

The polymers suitable for use with the present invention are substituted cellulosic polymers. By substituted cellulosic is meant that the polymer has a cellulosic backbone that has been modified by reaction of at least a portion of the hydroxyl groups on the saccharide repeating units with a compound to form an ester and/or an ether substituent.

The polymer is ionizable. By “ionizable” is meant that the polymer has ionizable substituents that are capable of being ionized at physiologically relevant pH of from 1-8. Preferred ionizable substituents include ether-linked alkyl carboxy groups, such as carboxy methyl and carboxy ethyl, and ester-linked substituents comprising a carboxylic acid group such as succinate, phthalate, and trimellitate. The ionizable substituents are present at a degree of substitution of at least 0.01, and more preferably at least 0.03. Nevertheless, the degree of substitution should not be so high as to render the polymer water soluble.

The polymer is poorly water soluble. By poorly water soluble is meant that when the polymer is administered alone at a solids concentration of 0.2 mg/mL to a phosphate buffered saline solution (PBS) at pH 6.5, the polymer has a solubility of 0.1 mg/mL or less. By “solids concentration” is meant the amount of solid polymer added to the PBS solution to measure the solubility of the polymer in the PBS solution. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. A test to determine whether the polymer is poorly water soluble may be performed as follows. The polymer is initially present in bulk form with average particle sizes of greater than 1 micron. The solid polymer alone is added to a PBS solution to achieve a solids concentration of 0.2 mg/mL polymer in the PBS solution. The PBS solution is stirred for approximately 1 hour at room temperature. Next, a nylon 0.45 μm filter is weighed, and the polymer solution is filtered. The filter is dried overnight at 40° C., and weighed the following morning. The amount of polymer dissolved is calculated from the amount of polymer added to the PBS solution minus the amount of polymer remaining on the filter (mg). Preferably, when administered at a solids concentration of 0.2 mg/mL to the PBS solution, the polymer has a solubility of less than 0.09 mg/mL, more preferably less than 0.07 mg/mL, more preferably less than 0.05 mg/mL, more preferably less than 0.03 mg/mL, and most preferably less than 0.01 mg/mL.

In order to be poorly water soluble, the polymer must generally have a sufficient number of hydrophobic groups relative to the ionizable groups. In a preferred embodiment, the poorly aqueous soluble ionizable cellulosic has an ether- or ester-linked alkyl substituent. Suitable alkyl substituents include C₁ to C₄ alkyl groups. Exemplary ether-linked alkyl substituents include methyl, ethyl, propyl, and butyl groups. Exemplary ester-linked alkyl substituents include acetate, propionate, and butyrate groups. In general, the alkyl substituent is present at a degree of substitution of at least 0.03. In one preferred embodiment, the alkyl substituent is present at a degree of substitution of at least 0.1, and more preferably at least 0.5.

Exemplary ionizable, poorly water soluble cellulosic polymers include: hydroxypropyl methyl cellulose acetate succinate, hydroxypropyl cellulose acetate succinate, hydroxypropyl methyl cellulose phthalate, carboxymethylethyl cellulose, cellulose acetate phthalate, cellulose acetate succinate, methyl cellulose acetate phthalate, hydroxypropyl methyl cellulose acetate phthalate, cellulose acetate trimellitate, hydroxypropyl methyl cellulose acetate trimellitate, ethyl cellulose succinate, ethyl cellulose phthalate, ethyl cellulose trimellitate, cellulose phthalate succinate, hydroxypropyl methyl cellulose phthalate succinate, hydroxypropyl methyl cellulose propionate trimellitate, hydroxypropyl methyl cellulose propionate phthalate, and hydroxypropyl methyl cellulose propionate succinate.

In one embodiment, the ionizable, poorly water soluble cellulosic polymer is selected from the group consisting of hydroxypropyl methyl cellulose acetate succinate (HPMCAS), hydroxypropyl methyl cellulose acetate phthalate (HPMCAP), hydroxypropyl methyl cellulose acetate trimellitate (HPMCAT), ethylcellulose succinate (ECS), ethylcellulose phthalate (ECP), ethylcellulose trimellitate (ECT), carboxymethyl ethylcellulose (CMEC), cellulose acetate propionate succinate (CAPrS), cellulose acetate succinate (CAS), cellulose propionate succinate (CPrS), cellulose acetate phthalate (CAP), and carboxymethylcellulose acetate butyrate (CMCAB).

In another embodiment, the ionizable, poorly water soluble cellulosic polymer is selected from the group consisting of HPMCAS, ECS, ECP, CAPrS, and CMCAB.

In another embodiment, the ionizable, poorly water soluble cellulosic polymer is HPMCAS.

In another embodiment, the ionizable, poorly water soluble cellulosic polymer is ECS.

In another embodiment, the ionizable, poorly water soluble cellulosic polymer is ECP.

In another embodiment, the ionizable, poorly water soluble cellulosic polymer is CAPrS.

In another embodiment, the ionizable, poorly water soluble cellulosic polymer is CMCAB.

In yet another embodiment, the ionizable, poorly water soluble cellulosic polymer comprises an ether-linked ethyl substituent, and an ether- or ester-linked ionizable substituent.

In still another embodiment, the ionizable, poorly water soluble cellulosic polymer is selected from the group consisting of ethylcellulose succinate, ethylcellulose phthalate, and ethylcellulose trimellitate.

Preferably, the polymer has limited water absorption. When equilibrated at 85% relative humidity at 25° C., the amount of water absorbed by the polymer is less than 7.5 wt %, and more preferably less than 7 wt %. Limiting the amount of water absorption by the polymer helps to improve the physically stability of the drug within the nanoparticle. Increasing amounts of water within the nanoparticle tend to lead to phase separation of the non-crystalline drug from the polymer, and/or crystallization. The water absorption may be less than 6.5 wt %, less than 6 wt %, or even less than 5 wt % when equilibrated at 85% relative humidity at 25° C.

### The Drug

The drug is a “poorly water soluble drug,” meaning that the drug has a solubility in water (over the pH range of 6.5 to 7.5 at 25° C.) of less than 5 mg/mL. The utility of the invention increases as the water solubility of the drug decreases. The drug may have an even lower solubility in water, such as less than about 1 mg/mL, less than about 0.1 mg/mL, and even less than about 0.01 mg/mL.

In general, it may be said that the drug has a dose-to-aqueous solubility ratio greater than about 10 mL, and more typically greater than about 100 mL, where the aqueous solubility (mg/mL) is the minimum value observed in any physiologically relevant aqueous solution (i.e., solutions with pH 1-8), including USP simulated gastric and intestinal buffers, and dose is in mg. Thus, a dose-to-aqueous solubility ratio may be calculated by dividing the dose (in mg) by the aqueous solubility (in mg/mL).

Preferred classes of drugs include, but are not limited to, compounds for use in the following therapeutic areas: antihypertensives, antianxiety agents, antiarrythmia agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, triglyceride-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-angiogenesis agents, anti-glaucoma agents, anti-depressants, and antiviral agents.

Each named drug should be understood to include the non-ionized form of the drug or pharmaceutically acceptable forms of the drug. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

Exemplary drugs suitable for use in the nanoparticles include, but are not limited to, phosphodiesterase inhibitors, such as sildenafil and sildenafil citrate; HMG-CoA reductase inhibitors, such as atorvastatin, lovastatin, simvastatin, pravastatin, fluvastatin, rosuvastatin, itavastatin, nisvastatin, visastatin, atavastatin, bervastatin, compactin, dihydrocompactin, dalvastatin, fluindostatin, pitivastatin, and velostatin (also referred to as synvinolin); vasodilator agents, such amiodarone; antipsychotics, such as ziprasidone; calcium channel blockers, such as nifedipine, nicardipine, verapamil, and amlodipine; cholesteryl ester transfer protein (CETP) inhibitors; cyclooxygenase-2 inhibitors; microsomal triglyceride transfer protein (MTP) inhibitors; vascular endothelial growth factor (VEGF) receptor inhibitors; carbonic anhydrase inhibitors; and glycogen phosphorylase inhibitors. Other low-solubility drugs suitable for use in the nanoparticles are disclosed in US Published patent application 2005/0031692, herein incorporated by reference.

In one embodiment, the drug is ziprasidone or a pharmaceutically acceptable form thereof.

In one embodiment, the drug is a hydrophobic non-ionizable drug. By “hydrophobic non-ionizable drug” is meant a subclass of non-ionizable drugs that are essentially water insoluble and highly hydrophobic, and are characterized by a set of physical properties, as described hereinafter. By “non-ionizable” is meant that the drug has substantially no ionizable groups. By “ionizable groups” is meant functional groups that are at least about 10% ionized over at least a portion of the physiologically relevant pH range of 1 to 8. Such groups have pKa values of about 0 to 9. Thus, hydrophobic non-ionizable drugs do not have a pKa value between 0 and 9.

The first property of hydrophobic drugs is that they are extremely hydrophobic. Log P, defined as the base 10 logarithm of the ratio of the drug solubility in octanol to the drug solubility in water, is a widely accepted measure of hydrophobicity. By “extremely hydrophobic” is meant that the Log P value of the drug is at least 4.0, preferably at least 4.5, and most preferably at least 5.0. Log P may be measured experimentally or calculated using methods known in the art. When using a calculated value for Log P, the highest value calculated using any generally accepted method for calculating Log P is used. Calculated Log P values are often referred to by the calculation method, such as C log P, A log P, and M log P. The Log P may also be estimated using fragmentation methods, such as Crippen's fragmentation method (27 J. Chem. Inf. Comput. Sci. 21 (1987)); Viswanadhan's fragmentation method (29 J. Chem. Inf. Comput. Sci. 163 (1989)); or Broto's fragmentation method (19 Eur. J. Med. Chem.-Chim. Theor. 71 (1984). Preferably the Log P value is calculated by using the average value estimated using Crippen's, Viswanadhan's, and Broto's fragmentation methods.

The second property of hydrophobic drugs is that they have an extremely low solubility in water over the pH range of 6.5 to 7.5 at 25° C. By “extremely low solubility in water” is meant that the solubility of the drug in water is less than 100 μg/mL. Preferably, the hydrophobic drug has a water solubility of less than 50 μg/mL, and most preferably less than 10 μg/mL.

In another embodiment the drug is a cholesteryl ester transfer protein (CETP) inhibitor. CETP inhibitors are drugs that inhibit CETP activity. The effect of a drug on the activity of CETP can be determined by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988, and as presented in U.S. Pat. No. 6,197,786, the disclosures of which are herein incorporated by reference. The potency of CETP inhibitors may be determined by performing the above-described assay in the presence of varying concentrations of the test compounds and determining the concentration required for 50% inhibition of transfer of radiolabeled lipids between lipoprotein fractions. This value is defined as the “IC₅₀ value.” Preferably, the CETP inhibitor has an IC₅₀ value of less than about 2000 nM, more preferably less than about 1500 nM, even more preferably less than about 1000 nM, and most preferably less than about 500 nM.

Specific examples of CETP inhibitors include [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester; (2R)-3-[[3-(4-chloro-3-ethylphenoxy)phenyl][[3-(1,1,2,2-tetrafluoroethoxy)phenyl]methyl]amino]-1,1,1-trifluoro-2-propanol; S-[2-([[1-(2-ethylbutyl)cyclohexyl]carbonyl]amino)phenyl]2-methylpropanethioate; trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid; trans-(4-{[N-(2-{[N′-[3,5-bis(trifluoromethyl)benzyl]-N′-(2-methyl-2H-tetrazol-5-yl)amino]methyl}-5-methyl-4-trifluoromethylphenyl)-N-ethylamino]methyl}cyclohexyl)acetic acid methanesulfonate; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; methyl N-(3-cyano-5-trifluoromethylbenzyl)-[6-(N′-cyclopentylmethyl-N′-ethylamino)indan-5-ylmethyl]-carbamate; methyl (3-cyano-5-trifluoromethylbenzyl)-[6-(N-cyclopentylmethyl-N-ethylamino)indan-5-ylmethyl]-carbamate; ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate; tert-butyl 5-(N-(3,5-bis(trifluoromethyl)benzyl)acetamido)-7-methyl-8-(trifluoromethyl)-2,3,4,5-tetrahydrobenzo[b]azepine-1-carboxylate; (3,5-bis-trifluoromethyl-benzyl)-(2-(cyclohexyl-methoxy-methyl)-5-trifluoromethyl-benzyl)-(2-methyl-2H-tetrazol-5-yl)-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine; the drugs disclosed in commonly owned U.S. patent application Ser. Nos. 09/918,127 and 10/066,091, the disclosures of both of which are incorporated herein by reference; and the drugs disclosed in the following patents and published applications, the disclosures of all of which are incorporated herein by reference: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; JP 11049743; WO 0018721; WO 0018723; WO 0018724; WO 0017164; WO 0017165; WO 0017166; EP 992496; EP 987251; WO 9835937; JP 03221376; WO 04020393; WO 05095395; WO 05095409; WO 05100298; WO 05037796; WO 0509805; WO 03028727; WO 04039364; WO 04039453; WO 0633002; and U.S. Provisional Patent Application Nos. 60/781,488 and 60/780,993, both of which were filed on Mar. 10, 2006.

Thus, in one embodiment, the CETP inhibitor is selected from the group of compounds mentioned above. In another embodiment, the CETP inhibitor is selected from the group consisting of (2R)-3-[[3-(4-chloro-3-ethylphenoxy)phenyl][[3-(1,1,2,2-tetrafluoroethoxy)phenyl]methyl]amino]-1,1,1-trifluoro-2-propanol; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexylyacetamide amine; (3,5-bis-trifluoromethyl-benzyl)-[2-(cyclohexyl-methoxy-methyl)-5-trifluoromethyl-benzyl]-(2-methyl-2H-tetrazol-5-yl)-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine; and pharmaceutically acceptable forms thereof.

In still another embodiment, the CETP inhibitor is (2R)-3-[[3-(4-chloro-3-ethylphenoxy)phenyl][[3-(1,1,2,2-tetrafluoroethoxy)phenyl]methyl]amino]-1,1,1-trifluoro-2-propanol.

In still another embodiment, the CETP inhibitor is trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide.

In another embodiment, the drug is an inhibitor of cyclooxygenase-2 (COX-2). COX-2 inhibitors are nonsteroidal anti-inflammatory drugs that exhibit anti-inflammatory, analgesic and antipyretic effects. Preferably, the COX-2 inhibitor is a selective COX-2 inhibitor, meaning that the drug is able to inhibit COX-2 without significant inhibition of cyclooxygenase-1 (COX-1). Preferably, the COX-2 inhibitor has a potency such that the concentration of drug that inhibits 50% of COX-2 enzyme in an in vitro test (i.e., the IC₅₀ value) is less than about 10 μM, preferably less than 5 μM, more preferably less than 2 μM. In addition, it is also preferable that the COX-2 inhibitor be selective relative to COX-1. Thus, preferably, the ratio of the IC_{50,COX-2} to IC_{50,COX-1} ratio for the compound is less than 0.5, more preferably less than 0.3, and most preferably less than 0.2.

Specific examples of COX-2 inhibitors include 4-(5-(4-methylphenyl)-3-(trifluoromethyl)-1H-pyrazol-1-yl)benzenesulfonamide (celecoxib); 4-(5-methyl-3-phenylisoxazol-4-yl)benzenesulfonamide (valdecoxib); N-(4-(5-methyl-3-phenylisoxazol-4-yl)phenylsulfonyl)propionamide (paracoxb); sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; 2-[(2-chloro-6-fluorophenyl)amino]-5-methyl benzeneacetic acid (lumiracoxib); 4-(3-(difluoromethyl)-5-(3-fluoro-4-methoxyphenyl)-1H-pyrazol-1-yl)benzenesulfonamide (deracoxib); 4-(4-(methylsulfonyl)phenyl)-3-phenylfuran-2(5H)-one (rofecoxib); 5-chloro-2-(6-methylpyridin-3-yl)-3-(4-(methylsulfonyl)phenyl)pyridine (etoricoxib); 2-(3,4-difluorophenyl)-4-(3-hydroxy-3-methylbutoxy)-5-(4-(methylsulfonyl)phenyl)pyridazin-3(2H)-one; (Z)-3-((3-chlorophenyl)(4-(methylsulfonyl)phenyl)methylene)-dihydrofuran-2(3H)-one, N-(2-(cyclohexyloxy)-4-nitrophenyl)methanesulfonamide; 4-Methyl-2-(3,4-dimethylphenyl)-1-(4-sulfamoyl-phenyl)-1H-pyrrole; 6-((5-(4-chlorobenzoyl)-1,4-dimethyl-1H-pyrrol-2-yl)methyl)pyridazin-3(2H)-one; 4-(4-cyclohexyl-2-methyloxazol-5-yl)-2-fluorobenzenesulfonamide (tilmacoxib); 2-(4-Ethoxyphenyl)-4-methyl-1-(4-sulfamoylphenyl)-1H-pyrrole; 4-hydroxy-2-methyl-N-(5-methyl-2-thiazolyl)-2H-1,2-benzothiazine-3-carboxamide-1,1-dioxide (meloxicam); 4-(4-chloro-5-(3-fluoro-4-methoxyphenyl)-1H-pyrazol-1-yl)benzenesulfonamide (cimicoxib), and pharmaceutically acceptable forms thereof; and the compounds disclosed in the following patents and published applications, the disclosures of which are incorporated herein by reference: U.S. Pat. No. 5,466,823, U.S. Pat. No. 5,633,272, U.S. Pat. No. 5,932,598, U.S. Pat. No. 6,034,256, U.S. Pat. No. 6,180,651, U.S. Pat. No. 5,908,858, U.S. Pat. No. 5,521,207, U.S. Pat. No. 5,691,374, WO 99/11605, WO 98/03484, and WO 00/24719.

Preferably the COX-2 inhibitor is selected from the group consisting of celecoxib; valdecoxib; paracoxb; sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; and pharmaceutically acceptable forms thereof. In one embodiment, the COX-2 inhibitor is celecoxib or pharmaceutically acceptable forms thereof.

### Process for Forming Nanoparticles

The nanoparticles may be formed by any process that results in formation of nanoparticles of non-crystalline drug dispersed in the polymer. The drug used to form the nanoparticles may be in a crystalline or non-crystalline form; however, at least 90 wt % of the drug in the resulting nanoparticles is in non-crystalline form.

One process for forming nanoparticles is a precipitation process. In this process, the drug and polymer are first dissolved in an organic solvent to form an organic solution. The organic solution is mixed with an aqueous solution in which the drug and polymer are poorly soluble, causing the nanoparticles to precipitate. Solvents suitable for forming the organic solution of dissolved drug and polymer can be any compound or mixture of compounds in which the drug and the polymer are mutually soluble and which is miscible in the aqueous solution. Preferably, the solvent is also volatile with a boiling point of 150° C. or less. Exemplary solvents include acetone, methanol, ethanol, tetrahydrofuran (THF), and DMSO. Mixtures of solvents, such as 50% methanol and 50% acetone, can also be used, as can mixtures with water, so long as the polymer and drug are sufficiently soluble to dissolve the drug and polymer. Preferred solvents are methanol, acetone, and mixtures thereof.

The amount of drug and polymer in the organic solution depends on the solubility of each in the solvent and the desired ratios of drug to polymer in the resulting nanoparticles. The organic solution may comprise from about 0.1 wt % to about 20 wt % dissolved solids. A dissolved solids content of from about 0.5 wt % to 10 wt % is preferred.

The aqueous solution may be any compound or mixture of compounds in which the drug and polymer are sufficiently insoluble so as to precipitate to form nanoparticles. The aqueous solution is preferably water.

The organic solution and aqueous solution are combined under conditions that cause the drug and polymer to precipitate as nanoparticles. The mixing can be by addition of a bolus or stream of organic solution to a stirring container of the aqueous solution. Alternately a stream or jet of organic solution can be mixed with a moving stream of aqueous solution.

The organic solution:aqueous solution volume ratio should be selected such that there is sufficient aqueous solution in the nanoparticle suspension that the nanoparticles solidify and do not rapidly agglomerate. However, too much aqueous solution will result in a very dilute suspension of nanoparticles, which may require further processing for ultimate use. Generally, the organic solution:aqueous solution volume ratio should be at least 1:100, but generally should be less than 1:2 (organic solution:aqueous solution). Preferably, the organic solution:aqueous solution volume ratio ranges from about 1:20 to about 1:3.

Once the nanoparticle suspension is made, a portion of the organic solvent may be removed from the suspension using methods known in the art. Exemplary processes for removing the organic solvent include evaporation, extraction, diafiltration, pervaporation, vapor permeation, distillation, and filtration. Preferably, the solvent is removed to a level that is acceptable according to ICH guidelines. Thus, the concentration of solvent in the nanoparticle suspension may be less than about 10 wt %, less than about 5 wt %, less than about 3 wt %, less than 1 wt %, and even less than 0.1 wt %.

An alternative process to form nanoparticles is an emulsification process. In this process, the drug and polymer are dissolved in an organic solvent that is immiscible with the aqueous solution. The organic solution is added to the aqueous solution and homogenized to form an emulsion of fine droplets of the water immiscible organic solution distributed throughout the aqueous phase. The organic solvent is then removed to form nanoparticles in the aqueous phase. Exemplary solvents for use in such a process include methylene chloride, ethyl acetate, and benzyl alcohol. The aqueous solution is preferably water.

The emulsion is generally formed by a two-step homogenization procedure. The organic solution of drug, polymer and organic solvent are first mixed with the aqueous solution using a rotor/stator or similar mixer to create a “pre-emulsion”. This mixture is then further processed with a high pressure homogenizer that subjects the droplets to very high shear, creating a uniform emulsion of very small droplets.

The volume ratio of organic solution to aqueous solution used in the process will generally range from 1:100 (organic solution:aqueous solution) to 2:3 (organic solution:aqueous solution). Preferably, the organic solution:aqueous solution volume ratio ranges from 1:9 to 1:2 (organic solution:aqueous solution).

A portion of the solvent is then removed forming a suspension of the nanoparticles in the aqueous solution. Exemplary processes for removing the solvent include evaporation, extraction, diafiltration, pervaporation, vapor permeation, distillation, and filtration. Preferably, the solvent is removed to a level that is acceptable according to The International Committee on Harmonization (ICH) guidelines, as described above.

Both the precipitation process and the emulsion process result in the formation of a suspension of the nanoparticles in the aqueous solution. In some instances it is desirable to concentrate the nanoparticles or to isolate the nanoparticles in solid form by removing some or all of the liquid from the suspension. Exemplary processes for removing at least a portion of the liquid include spray drying, spray coating, spray layering, lyophylization, evaporation, vacuum evaporation, filtration, ultrafiltration, reverse osmosis, and other processes known in the art. A preferred process is spray drying. One or more processes may be combined to remove the liquid from the nanoparticle suspension to yield a solid composition. For example, a portion of the liquids may be removed by filtration to concentrate the nanoparticles, followed by spray-drying to remove most of the remaining liquids, followed by a further drying step such as tray-drying.

When isolating the nanoparticles in solid form, it is often desirable to include a matrix material in the suspension of nanoparticles prior to removal of the liquids. The matrix material functions to help slow or prevent agglomeration of the nanoparticles as the liquids are being removed, as well as to help re-suspend the nanoparticles when the solid composition is added to an aqueous solution (e.g., an aqueous environment of use). The matrix material is preferably pharmaceutically acceptable and water soluble. Examples of matrix materials include polyvinyl pyrrolidone (PVP), trehalose, hydroxypropyl methyl cellulose (HPMC), hydroxypropyl cellulose (HPC), casein, caseinate, albumin, gelatin, acacia, lactose, mannitol, pharmaceutically acceptable forms thereof, and other matrix materials known in the art.

In one embodiment of the invention, a solid composition comprises (a) a plurality of nanoparticles comprising a poorly water-soluble drug and an ionizable, poorly water soluble cellulosic polymer, and (b) a matrix material. As used herein, the term “solid pharmaceutical composition” means that the composition is in a solid form and substantially free of liquids. The nanoparticles are entrapped or encapsulated in the matrix material.

The presence of nanoparticles in the solid composition can be determined using the following procedure. A sample of the solid composition is embedded in a suitable material, such as an epoxy or polyacrylic acid (e.g., LR White from London Resin Co., London, England). The sample is then microtomed to obtain a cross-section of the solid composition that is about 100 to 200 nm thick. This sample is then analyzed using transmission electron microscopy (TEM) with energy dispersive X-ray (EDX) analysis. TEM-EDX analysis quantitatively measures the concentration and type of atoms larger than boron over the surface of the sample. From this analysis, regions that are rich in drug can be distinguished from regions that are rich in the matrix material. The size of the regions that are rich in drug will have an average diameter of less than 500 nm in this analysis, demonstrating that the solid composition comprises nanoparticles of drug in the matrix material. See, for example, Transmission Electron Microscopy and Diffractometry of Materials (2001) for further details of the TEM-EDX method.

Another procedure that demonstrates the solid composition contains nanoparticles is to administer a sample of the solid composition to water to form a suspension of the nanoparticles. The suspension is then analyzed by DLS as described herein. A solid composition of the invention will form nanoparticles having an average cumulant diameter of less than 500 nm.

A specific procedure for demonstrating the solid composition contains nanoparticles is as follows. A sample of the solid composition is added to water at ambient temperature such that the concentration of solids is less than about 1 mg/mL. The so-formed suspension is then analyzed by DLS. The solid composition contains nanoparticles if the DLS analysis results in particles having an average cumulant diameter of less than 500 nm.

A solid composition of the invention will show the presence of nanoparticles in at least one, and preferably both of the above tests.

### Dosage Forms

The compositions of the present invention may be administered using any known dosage form. The nanoparticles may be formulated for administration via oral, topical, subdermal, intranasal, buccal, intrathecal, ocular, intraaural, intraarticular, subcutaneous spaces, vaginal tract, arterial and venous blood vessels, pulmonary tract or intramuscular tissue of an animal, such as a mammal and particularly a human. Oral dosage forms include: powders or granules; tablets; chewable tablets; capsules; unit dose packets, sometimes referred to in the art as “sachets” or “oral powders for constitution” (OPC); syrups; and suspensions. Parenteral dosage forms include depots, reconstitutable powders or suspensions. Topical dosage forms include creams, pastes, suspensions, powders, foams and gels. Ocular dosage forms include depots, emulsions, suspensions, powders, gels, creams, pastes, solid inserts and implants.

In one embodiment, the compositions of the present invention are capable of improving the concentration of dissolved drug in a use environment relative to a control composition consisting essentially of the drug alone without any of the polymer. In order to determine concentration enhancement in vitro, the amount of “free” drug, or solvated drug is measured. By “free” drug is meant drug which is in the form of dissolved drug or present in micelles, but which is not in the nanoparticles or any solid particles larger than 500 nm, such as precipitate. A composition of the invention provides concentration enhancement if, when administered to an aqueous use environment, it provides a free drug concentration that is at least 1.25-fold the free drug concentration provided by the control composition. Preferably, the free drug concentration provided by the compositions of the invention are at least about 1.5-fold, more preferably at least about 2-fold, and most preferably at least about 3-fold that provided by the control composition.

Alternatively, the compositions of the present invention, when dosed orally to a human or other animal, provide an AUC in drug concentration in the blood plasma or serum (or relative bioavailability) that is at least 1.25-fold that observed in comparison to the control composition. Preferably, the blood AUC is at least about 2-fold, more preferably at least about 3-fold, even more preferably at least about 4-fold, still more preferably at least about 6-fold, yet more preferably at least about 10-fold, and most preferably at least about 20-fold that of the control composition. The determination of AUCs is a well-known procedure and is described, for example, in Welling, “Pharmacokinetics Processes and Mathematics,” ACS Monograph 185 (1986).

Alternatively, the compositions of the present invention, when dosed orally to a human or other animal, provide a maximum drug concentration in the blood plasma or serum (Cₘₐₓ) that is at least 1.25-fold that observed in comparison to the control composition. Preferably, the Cₘₐₓ is at least about 2-fold, more preferably at least about 3-fold, even more preferably at least about 4-fold, still more preferably at least about 6-fold, yet more preferably at least about 10-fold, and most preferably at least about 20-fold that of the control composition. Thus, compositions that meet the in vitro or in vivo performance criteria, or both, are considered to be within the scope of this embodiment.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that variations of the conditions and processes of the following examples can be used.

### EXAMPLES

### Preparation of Polymers

### HPMCAS

Polymer 1, hydroxypropyl methylcellulose acetate succinate (HPMCAS), having the degree of substitution shown in Table 1, was synthesized using the following procedure. First, 99.9 g of glacial acetic acid was added to a 250 mL round bottom flask equipped with a water condenser and a stir bar and placed into an oil bath set at 86° C. The flask was purged with nitrogen. To this, 10.025 g of HPMC (Dow E3 Prem LV, having a calculated DOS_{M} of 1.88 and a DOS_{HP} of 0.25 according to the wt % from the certificate of analysis provided by the manufacturer) and 10.100 g of sodium acetate were added and allowed to dissolve. Once complete dissolution of the HPMC occurred, 0.3839 g of succinic anhydride was added and the mixture was allowed to react for 6 hours. Next, 40.100 g acetic anhydride was added and the mixture was allowed to react for 18 hours.

The reaction mixture was quenched into about 800 mL of water, precipitating the polymer. The polymer was filtered using a Buchner funnel, washed with water, and redissolved in acetone. The polymer was precipitated again in about 750 mL water, filtered, washed with water, and dried in vacuo to yield an off-white solid.

The degree of acetate and succinate substitution on the polymer was determined and the results are given in Table 1. The degree of substitution for methoxy and hydroxypropoxy were assumed to be unchanged from the certificate of analysis provided by the manufacturer of the HPMC starting material.

Polymer 2 was prepared with the degrees of substitution given in Table 1, using the following procedure. First, a 250 mL round bottom flask equipped with a water condenser and a stir bar, was placed in a heating mantle and purged with nitrogen for about 5 minutes. Next, 50 mL of pyridine was added. To this, 2.5027 g of HPMC (SE Pharmacoat 904) was added and allowed to dissolve. Once complete dissolution of the HPMC occurred, 1.093 g of acetic anhydride was added and the mixture was allowed to react for about 2½ hours. Next, 0.7518 g succinic anhydride was added and the mixture was allowed to react for about 3½ hours. The reaction mixture was quenched into about 700 mL of water, precipitating the polymer. Next, 100 mL 50:50 concentrated HCl:water was added to reduce the pH to about 5. The polymer was filtered using a Buchner funnel, washed with water, and redissolved in acetone. The polymer was precipitated again in about 500 mL water, filtered, washed with water, and dried in vacuo to yield an off-white solid. The T_{g} was also determined using differential scanning calorimetry (DSC) at 0% RH, and the data is included in Table 1.

TABLE 1

| | | | | | Total | T_{g} |
|---|---|---|---|---|---|---|
| Polymer | DOS_{HP} | DOS_{M} | DOS_{Ac} | DOS_{suc} | DOS | (° C. at <5% RH) |
| 1 | 0.25 | 1.88 | 0.77 | 0.05 | 2.70 | ND* |
| 2 | 0.17 | 1.43 | 1.39 | 0.07 | 2.89 | 138 |

*ND = not determined

### ECP and ECS

Polymer 3, ethylcellulose phthalate (ECP), was synthesized using the following procedure. First, 100 mL of pyridine was added to a 250 mL round bottom flask equipped with a water condenser and a stir bar and placed into an oil bath set at 85° C. The flask was purged with argon. To this, 12.207 g of ethylcellulose (ETHOCEL, standard viscosity grade 4 available from Dow Chemical Co., Midland, Mich.) was added and allowed to dissolve. Once complete dissolution of the ETHOCEL occurred, 20.005 g of phthalic anhydride was added and the mixture was allowed to react for 6 hours.

The reaction mixture was quenched into about 800 mL of water containing 100 mL concentrated HCl, precipitating the polymer. Additional HCl was added to obtain a pH of 3, and the suspension was blended in a blender. The polymer was filtered using a Buchner funnel, then blended and precipitated again in about 800 mL water, filtered, and air dried (ambient conditions).

Polymer 4, ethylcellulose succinate (ECS), was synthesized using the following procedure. First, 200 mL of glacial acetic acid was added to a round bottom flask equipped with a water condenser and a stir bar and placed into an oil bath set at 85° C. The flask was purged with argon. To this, 20.011 g of ETHOCEL and 20.028 g of sodium acetate were added and allowed to dissolve. Once complete dissolution of the ETHOCEL occurred, 15.001 g of succinic anhydride was added and the mixture was allowed to react overnight.

The reaction mixture was quenched into about 1600 mL of water, and the suspension was blended in a blender. The polymer was filtered using a Buchner funnel, then blended and precipitated again in about 1600 mL water. The polymer was filtered and precipitated a third time in about 1600 mL hot water. The precipitate was filtered and dried in vacuo to yield an off-white solid.

### CMCAB

Polymer 5 in the examples below is carboxymethyl cellulose acetate butyrate (CMCAB-641-0.5 available from Eastman Chemical Company, Kingsport, Tenn.).

### CAPrS

Polymer 6, cellulose acetate propionate succinate (CAPrS), was synthesized using the following procedure. First, 200 mL of pyridine was added to a round bottom flask equipped with a water condenser and a stir bar and the solvent was heated to reflux. To this, 10.037 g of cellulose acetate propionate (CAPr, 0.6% acetate and 42.5% propionate, available from Sigma-Aldrich Corp., St. Louis, Mo.) was added and allowed to dissolve. Once complete dissolution of the CAPr occurred, 12.50 g of succinic anhydride was added and the mixture was stirred at reflux for 6 hours.

The reaction mixture was quenched into about 2 L of water containing 300 mL concentrated HCl, precipitating the polymer. The polymer was filtered using a Buchner funnel, then rinsed with about 800 mL water. The filtrate was dried in vacuo to yield an off-white solid.

### Polymer Screening Examples

The degree of water solubility of Polymers 2, 3, and 5 were evaluated as follows. First, a sufficient amount of solid polymer was added to a phosphate buffered saline (PBS) solution so that the solids concentration of polymer in the PBS solution was 0.2 mg/mL for each respective polymer. The PBS solution comprised 20 mM Na₂HPO₄, 47 mM KH₂PO₄, 87 mM NaCl, and 0.2 mM KCl, and was adjusted to pH 6.5 with NaOH. The polymer was stirred in PBS solution for approximately 1 hour at room temperature. Next, a nylon 0.45 μm filter was weighed, and the polymer solution was filtered. The filter was dried overnight at 40° C., and weighed the following morning. The amount of dissolved polymer was calculated from amount of polymer added to the PBS solution minus the amount of polymer remaining on the filter (mg). The results of these tests on the polymers above, used in the examples below, are shown in Table 2.

TABLE 2

| Example | Polymer | Solubility (mg/mL) at | |
|---|---|---|---|
| Polymer | Number | pH 6.5 | Observations |
| HPMCAS | 2 | 0.02 | cloudy |
| ECP | 3 | 0.1 | cloudy |
| CMCAB | 5 | 0.02 | cloudy |

### Control Polymers

Commercially available enteric cellulosic polymers were tested for comparison to the polymers of the invention. The screening procedure above was used to test the control polymers, and the results are shown in Table 3. Control Polymer 1, “CP1”, is hydroxypropyl methylcellulose acetate succinate, HPMCAS-HF, (AQOAT-HF, available from Shin Etsu). Control Polymer 2, “CP2”, is hydroxypropyl methylcellulose acetate succinate, HPMCAS-MF, (AQOAT-MF, available from Shin Etsu). Control Polymer 3, “CP3”, is hydroxypropyl methylcellulose phthalate, HPMCP, (available from Shin Etsu). Control Polymer 4, “CP4”, is cellulose acetate phthalate, CAP, (available from Spectrum Chemical). Control Polymer 5, “CP5”, is cellulose acetate trimellitate, CAT, (available from Aldrich Chemical). Control Polymer 6, “CP6”, is carboxy methyl ethyl cellulose, CMEC, (available from Freund).

TABLE 3

| | Control | | |
|---|---|---|---|
| | Polymer | Solubility (mg/mL) at | |
| Control Polymer | Number | pH 6.5 | Observations |
| HPMCAS-HF | CP1 | 0.2 | clear |
| HPMCAS-MF | CP2 | 0.2 | clear |
| HPMCP | CP3 | 0.2 | clear |
| CAP | CP4 | 0.2 | clear |
| CAT | CP5 | 0.2 | clear |
| CMEC | CP6 | 0.2 | clear |

These commercially available enteric cellulosic polymers are not poorly water soluble as shown in Table 3.

The degree of water absorption was also determined for the polymers as follows. Dynamic vapor sorption (DVS) analysis was performed on the polymers described above, using a Surface Measurement Systems DVS-1000. For DVS analysis, a sample (10-50 mg) was weighed into a sample pan, and the humidity was equilibrated to 0° C. at 25° C. An initial weight was measured, and the humidity was increased from 0 to 90% RH in increments. At each increment, the sample weight was measured and the weight percent water absorbed was calculated. The degree of water absorption at 85% relative humidity and 25° C. is shown in Table 4 below.

TABLE 4

| | Polymer | Water absorption at 85% RH |
|---|---|---|
| Polymer | Number | (wt %) |
| HPMCAS | 1 | 6.8 |
| HPMCAS | 2 | 7.0 |
| ECP | 3 | 3.8 |
| ECS | 4 | 4.7 |
| CMCAB | 5 | 5.3 |
| HPMCAS-HF | CP1 | 7.7 |
| HPMCAS-MF | CP2 | 8.1 |

### Drugs Used in Examples

The following drugs were used in the examples described below.

Drug 1 was 4-[5-(4-methylphenyl)-3-(trifluoromethyl)-1H-pyrazol-1-yl]benzenesulfonamide, also known as celecoxib, having the structure:

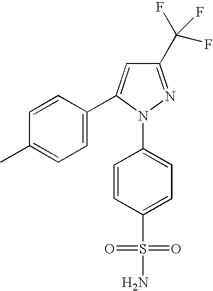

Drug 1 has a solubility in water of about 3.5 μg/mL, and a Log P value of 3.75. The T_{g} of amorphous Drug 1 was determined by DSC analysis to be 54° C.

Drug 2 was [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester, having the structure:

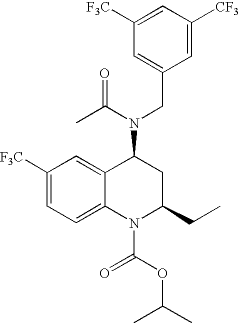

Drug 2 has a solubility in water of less than 10 ng/mL, and a C Log P value of about 6.6. The T_{g} of amorphous Drug 2 was determined by DSC analysis to be about 45° C., and the Tₘ is about 111° C.

Drug 3 was diphenylhydantoin (phenyloin) having the structure:

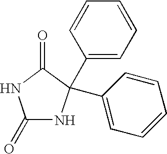

Drug 3 has a solubility in water at pH 7.5 of about 22 μg/mL, and has a Log P value of 2.47.

Drug 4 was 5-(2-(4-(3-benzisothiazolyl)-piperazinyl)ethyl-6-chlorooxindole, also known as ziprasidone (free base), having the structure:

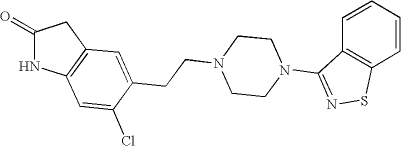

The solubility of Drug 4 in water is about 0.15 μg/mL. Drug 4 has a Log P value of about 3.6. The Tₘ of Drug 4 is about 223° C.

### Examples 1 and 2

Nanoparticles of Drug 1 with Polymers 1 and 2 were prepared using a precipitation process as follows (composition and amounts shown in Table 5). First, a water-miscible organic solution was formed by mixing 5 mg celecoxib (Drug 1) and 15 mg HPMCAS (Polymer 1 and Polymer 2, respectively) in 2 mL acetone. The organic solution was mixed using a vortex mixer for 10 seconds. The aqueous buffer solution consisted of 9 mL of 50 mM KH₂PO₄, pH 6.5, in a stirred glass vial. To form the nanoparticles, a glass pipette containing the organic solution was inserted under the surface of the aqueous solution, and delivered into the stirring vortex all at once, rapidly precipitating the nanoparticles. The acetone was removed from the suspension using a rotary evaporator (room temperature, 200 rpm, 15 min), resulting in an aqueous suspension of nanoparticles.

### Examples 3-15

Nanoparticles were prepared with Drugs 1-4, and Polymers 2-6 using the following procedure (compositions and amounts shown in Table 5). First, a water-miscible organic solution was formed by mixing the drug and polymer in 8 mL acetone. The organic solution was filtered with a 1 μm glass filter. The aqueous solution consisted of 9 mL of filtered water, in a glass vial stirred at 400 rpm. To form the nanoparticles, a glass pipette containing 1 mL of the organic solution was inserted under the surface of the aqueous solution, and delivered into the stirring vortex all at once, rapidly precipitating the nanoparticles. The acetone was removed from the suspension using a rotary evaporator (room temperature, 200 rpm, 15 min), resulting in an aqueous suspension of nanoparticles. Drug and polymer amounts in the organic solution were varied as noted in Table 5.

TABLE 5

| | | Drug | | Polymer | |
|---|---|---|---|---|---|
| | Formulation | amount | | amount | Acetone |
| Sample | (polymer number) | (mg) | Polymer | (mg) | (mL) |
| Example 1 | 25% Drug 1: HPMCAS (1) | 5 | 1 | 15 | 2 |
| Example 2 | 25% Drug 1: HPMCAS (2) | 5 | 2 | 15 | 2 |
| Example 3 | 25% Drug 1: HPMCAS (1) | 20.3 | 1 | 60.3 | 8 |
| Example 4 | 25% Drug 1: HPMCAS (2) | 20.1 | 2 | 59.8 | 8 |
| Example 5 | 25% Drug 2: HPMCAS (1) | 19.9 | 1 | 59.7 | 8 |
| Example 6 | 25% Drug 2: HPMCAS (2) | 20.2 | 2 | 60.1 | 8 |
| Example 7 | 25% Drug 2: ECP (3) | 20.0 | 3 | 59.8 | 8 |
| Example 8 | 25% Drug 2: ECS (4) | 20.0 | 4 | 59.5 | 8 |
| Example 9 | 25% Drug 1: CMCAB (5) | 20.0 | 5 | 59.6 | 8 |
| Example 10 | 25% Drug 2: CMCAB (5) | 19.6 | 5 | 59.7 | 8 |
| Example 11 | 25% Drug 3: CMCAB (5) | 20.0 | 5 | 59.9 | 8 |
| Example 12 | 25% Drug 4: CMCAB (5) | 20.2 | 5 | 59.9 | 8 |
| Example 13 | 25% Drug 1: CAPrS (6) | 20.0 | 6 | 60.0 | 8 |
| Example 14 | 25% Drug 2: CAPrS (6) | 20.5 | 6 | 60.8 | 8 |
| Example 15 | 25% Drug 2: ECS (4) | 5 | 4 | 15 | 2 |
| Control 1 | 25% Drug 1: HPMCAS-HF (CP2) | 5 | CP2 | 15 | 2 |
| Control 2 | 25% Drug 2: HPMCAS-MF (CP2) | 5 | CP2 | 15 | 2 |

### Example 15

Nanoparticles were prepared with Drug 2 and Polymer 4 using the procedure described for Examples 1 and 2. Drug and polymer amounts in the organic solution were varied as noted in Table 5. For this example, 2 mL organic solution was added to 9 mL aqueous buffer solution (pH 6.5).

### Control 1

The nanoparticles of Control 1 were prepared with Drug 1 and water soluble HPMCAS (CP2) using the procedure described for Examples 1 and 2. Drug and polymer amounts in the organic solution were varied as noted in Table 5.

### Control 2

The nanoparticles of Control 2 were prepared with Drug 2 and Control Polymer 2 (CP2) using the procedure described for Examples 1 and 2. Drug and polymer amounts were varied as noted in Table 5.

### Dynamic Light Scattering Analysis

For dynamic light scattering (DLS) analysis, the aqueous suspensions were filtered using a 1 μm glass membrane filter (Anatop filter, Watman), and poured into a cuvette. Dynamic light-scattering was measured using a Brookhaven Instruments BI-200SM particle size analyzer with a BI-9000AT correlator. The size of the nanoparticles (cumulant) of Examples 1-15, and Controls 1 and 2, are shown in Table 6.

TABLE 6

| | | Initial | |
|---|---|---|---|
| | | Cumulant | |
| | Formulation | Particle | |
| Sample | (polymer number) | Size (nm) | Polydispersity |
| Example 1 | 25% Drug 1: HPMCAS (1) | 96 | 0.12 |
| Example 2 | 25% Drug 1: HPMCAS (2) | 122 | 0.33 |
| Example 3 | 25% Drug 1: HPMCAS (1) | 92 | 0.07 |
| Example 4 | 25% Drug 1: HPMCAS (2) | 82 | 0.10 |
| Example 5 | 25% Drug 2: HPMCAS (1) | 138 | 0.14 |
| Example 6 | 25% Drug 2: HPMCAS (2) | 81 | 0.15 |
| Example 7 | 25% Drug 2: ECP (3) | 267 | 0.16 |
| Example 8 | 25% Drug 2: ECS (4) | 119 | 0.15 |
| Example 9 | 25% Drug 1: CMCAB (5) | 101 | 0.12 |
| Example 10 | 25% Drug 2: CMCAB (5) | 99 | 0.16 |
| Example 12 | 25% Drug 4: CMCAB (5) | 79 | 0.18 |
| Example 13 | 25% Drug 1: CAPrS (6) | 95 | 0.14 |
| Example 14 | 25% Drug 2: CAPrS (6) | 88 | 0.16 |
| Example 15 | 25% Drug 2: ECS (4) | 133 | 0.36 |
| Control 1 | 25% Drug 1: HPMCAS (CP2) | 142 | 0.23 |
| Control 2 | 25% Drug 2: HPMCAS (CP2) | 372 | 0.14 |

### Nanoparticle Suspension Stability

The aqueous nanoparticle suspensions of Examples 1, and 2, and Control 1, were filtered using a 1 μm glass membrane filter and allowed to stand unmixed (ambient conditions) to measure stability. DLS analysis showed that the volume-weighted mean diameter of the nanoparticles in suspension remained small for at least 2 days. These results are shown in Table 7.

TABLE 7

| | | | | Particle |
|---|---|---|---|---|
| | | Initial | Storage | size after |
| | Formulation | Particle | duration | storage |
| Sample | (polymer number) | Size (nm) | (days) | (nm) |
| Example 1 | 25% Drug 1: HPMCAS (1) | 96 | 2 | 98 |
| Example 2 | 25% Drug 1: HPMCAS (2) | 122 | 2 | 118 |
| Control 1 | 25% Drug 1: HPMCAS | 142 | 2 | 169 |
| | (CP2) | | | |

### Potency

Stability was also evaluated by measuring potency of the suspensions over time. To measure nanoparticle potency, a sample of the aqueous nanoparticle suspensions of Examples 1 and 2, and Control 1, was taken at 1, 2, and 5 days after formation of the suspensions. The samples were filtered using a 1 μm glass membrane filter and diluted in methanol. The concentration of drug in the filtered samples was analyzed by high-performance liquid chromatography (HPLC).

For Drug 1, HPLC analysis was performed using a Zorbax SB C₈ column. The mobile phase consisted of 55% acetonitrile/45% 5 mM triethanolamine, adjusted to pH 7.0. UV absorbance was measured at 254 nm.

Potencies of the nanoparticle suspensions are shown in Table 8. The results in Table 8 show that the nanoparticle suspensions of the invention maintained greater than 70% potency for at least 5 days, indicating that the nanoparticles remained in solution as submicron particles. In contrast, the control suspensions showed a significant loss in potency over time, indicating that the nanoparticles aggregated into particles with sizes greater than one micron.

TABLE 8

| | | Potency | | |
|---|---|---|---|---|
| | | Day 1 | Potency | Potency |
| | Formulation | (mgA/ | Day 2 | Day 5 |
| Sample | (polymer number) | mL)* | (mgA/mL) | (mgA/mL) |
| Example 1 | 25% Drug 1: HPMCAS (1) | 0.25 | 0.21 | 0.18 |
| Example 2 | 25% Drug 1: HPMCAS (2) | 0.21 | 0.17 | 0.15 |
| Control 1 | 25% Drug 1: HPMCAS | 0.22 | 0.17 | 0.03 |
| | (CP2) | | | |

*“mgA” refers to mg of active drug.

### Optical Microscopy

After 6 days, the nanoparticle suspensions of Examples 1 and 2, and Control 1, were observed at 1000× using optical microscopy. The Example 1 suspension was slightly cloudy with no crystals and some visible precipitate. The Example 2 suspension was mostly clear with no crystals and some visible precipitate. For Control 1, the suspension appeared cloudy, with many crystals observed. This indicates that the nanoparticle suspensions of the invention were more stable than the control.

### Examples 16 and 17

Nanoparticles were prepared with Drug 2 and Polymer 4 using the procedure described for Examples 3-15. Drug and polymer amounts in the organic solution were varied as noted in Table 9. For the nanoparticles of Example 16, 16 mL organic solution was added to 144 mL water. For the nanoparticles of Example 17, 1 mL organic solution was added to 9 mL water.

TABLE 9

| | | Drug | | Polymer | |
|---|---|---|---|---|---|
| | | amount | | amount | Acetone |
| Sample | Formulation | (mg) | Polymer | (mg) | (mL) |
| Example | 25% Drug 2: ECS (4) | 40 | 4 | 120 | 16 |
| 16 | | | | | |
| Example | 50% Drug 2: ECS (4) | 5 | 4 | 5 | 1 |
| 17 | | | | | |

### Dynamic Light Scattering analysis

The results of dynamic light scattering showing particle size are shown in Table 10.

TABLE 10

| | | Cumulant |
|---|---|---|
| | Formulation | Particle Size |
| Sample | (polymer number) | (nm) |
| Example 16 | 25% Drug 2: ECS (4) | 165 |
| Example 17 | 50% Drug 2: ECS (4) | 238 |

### Cryo-Transmission Electron Microscope analysis

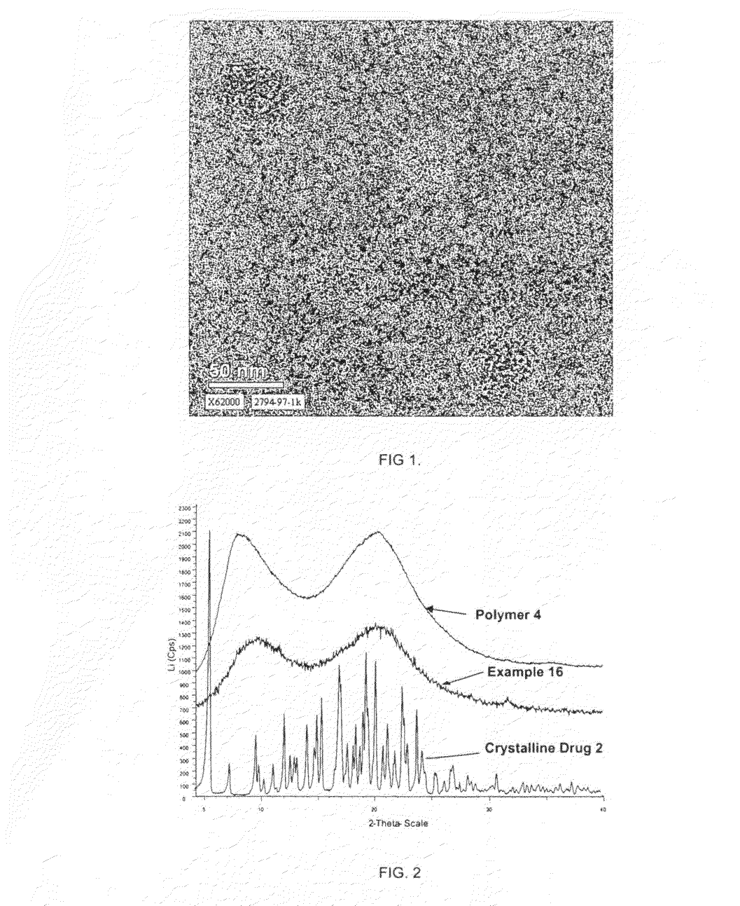

The nanoparticles of Example 16 were evaluated using cryo-transmission electron microscopy (TEM). A Tecnai series TEM (FEI Company; Hillsboro, Oreg.) was used to observe the fine structure morphology of nanoparticles in the aqueous suspension. Results are shown in FIG. 1.

### Zeta Potential Analysis

The zeta potential of the nanoparticles of Example 16 was measured using a Brookhaven Instruments particle size analyzer with Phase Analysis Light Scattering (“ZetaPALS”). The zeta potential was found to be −23 mV, indicating that the ionizable groups on Polymer 4 migrated to the surface to provide small, stable nanoparticles.

### Free Drug Measurement

The amount of free drug provided by suspensions of the nanoparticles of Examples 16 and 17 was measured. The nanoparticle suspensions of Examples 16 and 17 were added to phosphate-buffered saline, pH 6.5, containing 2 wt % of a 4/1 mixture of sodium taurocholic acid and 1-palmitoyl-2-oleyl-sn-glycero-3-phosphocholine (NaTC/POPC), for a final concentration of 30 mgA/mL. This solution was filtered using a centrifuge tube filter with a 30,000-dalton molecular-weight cutoff. The filtrate solution was assayed by high-performance liquid chromatography (HPLC). The free drug provided by crystalline drug was also determined using the same procedure. The nanoparticles of Example 16 provided a free drug concentration of about 0.99 fold that provided by crystalline drug, while the nanoparticles of Example 17 provided a free drug concentration of about 1.9 fold that provided by crystalline drug. The results show that the amount of free drug provided may be controlled by varying the ratio of drug to polymer, and that the concentration of free drug provided by the nanoparticles may be enhanced relative to crystalline drug.

### Isolation of Solid Nanoparticles

The nanoparticles of Example 16 were spray-dried by pumping the aqueous suspension into a small-scale spray-drying apparatus at a rate of 0.2 ml/min using a syringe pump. The heated gas entered the chamber at an inlet temperature of 120° C., with a flow of 1 SCFM. The resulting solid nanoparticles were collected for PXRD analysis.

### Powder X-Ray Diffraction analysis

The spray-dried nanoparticles of Example 16 were examined by PXRD with a Bruker AXS D8 Advance diffractometer to determine the amorphous character of the drug in the nanoparticles. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The x-ray source (KCu_{α}, λ=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 2θ range of 4° to 40°. Crystalline Drug 2 and Polymer 4 were also analyzed using the same procedure for comparison. The results are shown in FIG. 2, where the diffractograms have been shifted relative to each other for clarity. The nanoparticles of Example 16 exhibited a diffraction pattern showing only an amorphous halo, while crystalline Drug 2 exhibited a pattern showing sharp peaks characteristic of crystalline drug. These data indicate that the drug in the nanoparticles of Example 16 is amorphous and not crystalline.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A pharmaceutical composition comprising nanoparticles, said nanoparticles comprising:
(a) a drug having a solubility in water of less than 5 mg/mL over the pH range of 6.5 to 7.5 at 25° C., at least 90 wt % of said drug being non-crystalline; and
(b) an ionizable cellulosic polymer having a water solubility such that when said polymer is administered alone at a solids concentration of 0.2 mg/mL to a phosphate buffered saline solution consisting of an aqueous solution of 20 mM sodium phosphate (Na₂HPO₄), 47 mM potassium phosphate (KH₂PO₄), 87 mM NaCl, and 0.2 mM KCl, adjusted to pH 6.5 with NaOH, said polymer has a solubility of 0.1 mg/mL or less
wherein said nanoparticles have an average size of less than 500 nm and said drug and said polymer constitute at least 60 wt % of said nanoparticles.

2. The composition of claim 1 wherein said polymer, when equilibrated at 85% relative humidity at 25° C., absorbs less than 7.5 wt % water.

3. The composition of claim 1 wherein said drug and said polymer are molecularly interdispersed.

4. The composition of claim 1 wherein said nanoparticles comprise a solid solution of said drug and said polymer.

5. The composition of claim 1 wherein said nanoparticles have an average size of less than 300 nm.

6. The composition of claim 5 wherein said nanoparticles have an average size of less than 150 nm.

7. The composition of claim 1 wherein said nanoparticles have a zeta potential with an absolute value of greater than 10 mV.

8. The composition of claim 1 wherein said drug and said polymer constitute at least 80 wt % of said nanoparticles.

9. The composition of claim 1 wherein said nanoparticles consist essentially of said drug and said polymer.

10. The composition of claim 1 wherein said nanoparticles are substantially free from a surfactant.

11. The composition of claim 1 wherein said polymer comprises an ether-linked ethyl substituent and an ether- or ester-linked ionizable substituent.

12. The composition of claim 1 wherein said polymer is selected from the group consisting of hydroxypropyl methyl cellulose acetate succinate, hydroxypropyl cellulose acetate succinate, hydroxypropyl methyl cellulose phthalate, carboxymethylethyl cellulose, cellulose acetate phthalate, cellulose acetate succinate, methyl cellulose acetate phthalate, hydroxypropyl methyl cellulose acetate phthalate, cellulose acetate trimellitate, hydroxypropyl methyl cellulose acetate trimellitate, ethyl cellulose succinate, ethyl cellulose phthalate, ethyl cellulose trimellitate, cellulose phthalate succinate, hydroxypropyl methyl cellulose phthalate succinate, hydroxypropyl methyl cellulose propionate trimellitate, hydroxypropyl methyl cellulose propionate phthalate, and hydroxypropyl methyl cellulose propionate succinate.

13. The composition of claim 1 wherein said polymer is selected from the group consisting of hydroxypropyl methyl cellulose acetate succinate (HPMCAS), hydroxypropyl methyl cellulose acetate phthalate (HPMCAP), hydroxypropyl methyl cellulose acetate trimellitate (HPMCAT), ethylcellulose succinate (ECS), ethylcellulose phthalate (ECP), ethylcellulose trimellitate (ECT), carboxymethyl ethylcellulose (CMEC), cellulose acetate propionate succinate (CAPrS), cellulose acetate succinate (CAS), cellulose propionate succinate (CPrS), cellulose acetate phthalate (CAP), and carboxymethylcellulose acetate butyrate (CMCAB).

14. The composition of claim 1 wherein said polymer is selected from the group consisting of ethylcellulose succinate, ethylcellulose phthalate, and ethylcellulose trimellitate.

15. The composition of claim 1 wherein said solubility of said polymer is less than 0.07 mg/mL.

16. The composition of claim 1 wherein said solubility of said polymer is less than 0.05 mg/mL.

17. The composition of claim 1 wherein said polymer has a degree of substitution of ionizable substituents of at least 0.03.

18. The composition of claim 1 wherein said solubility of said drug is less than 1 mg/mL.

19. The composition of claim 1 further comprising a matrix material, wherein said nanoparticles are entrapped in said matrix material.

20. The composition of claim 19 wherein said matrix material is selected from the group consisting of polyvinyl pyrrolidone (PVP), trehalose, hydroxypropyl methyl cellulose (HPMC), hydroxypropyl cellulose (HPC), casein, caseinate, albumin, gelatin, acacia, lactose, mannitol, pharmaceutically acceptable forms thereof, and mixtures thereof.
